(19) United States
(12) Patent Application Publication
(10) Pub. No.: US 2007/0037873 A1
(43) Pub. Date: Feb. 15, 2007

(54) Airway remodeling treatments

(76) Inventors: Robert B. Zurier, Princeton, MA (US); J. Mark Madison, Holden, MA (US)

Correspondence Address: FISH & RICHARDSON PC, P.O. BOX 1022, MINNEAPOLIS, MN, 55440-1022, US

(21) Appl. No.: 11/500,580

(22) Filed: Aug. 8, 2006

Related U.S. Application Data
(60) Provisional application No. 60/706,272, filed on Aug. 8, 2005.

(51) Int. Cl. A61K 31/353

(52) U.S. Cl. 514/454

(57) ABSTRACT

Described are new uses for ajulemic acid and derivatives of ajulemic acid to treat airway remodeling.

### CROSS-REFERENCE TO RELATED APPLICATIONS

This application claims priority to U.S. Provisional Patent Application Ser. No. 60/706,272, filed on Aug. 8, 2005, the contents of which is hereby incorporated by reference in its entirety.

### TECHNICAL FIELD

This invention relates to methods of using ajulemic acid and derivatives of ajulemic acid to treat airway remodeling associated with asthma.

### BACKGROUND

Asthma is characterized by the occurrence of short-term, reversible airflow obstructions, i.e., asthma attacks. However, recent evidence indicates that many asthmatics also exhibit a residual and persistent airway obstruction that is associated with long-term alteration of the airway structure. This persistent alteration of airway structure is also known as airway remodeling. Structural alterations in airway tissue include one or more of the following: thickening of the reticular basement membrane in the asthmatic bronchus, abnormal elastic fiber network in the bronchial interstitial matrix, hyperplasia of collagen fibers in submucosa, altered vascular morphology, proliferation of mucous glands, and the hypertrophy and hyperplasia of smooth muscle cells. See, e.g., Buhl and Farmer, Proc. Am. Thorac. Soc., 1:136-142, 2004 and Vignola et al., Chest, 123(Supp):417S-422S, 2003.

Generally, current asthma medications include short-term bronchodilators, long-term bronchodilators, and anti-inflammatories. Short-term bronchodilators quickly relax the constriction of airway muscle cells, thereby alleviating obstructed airflow in a patient suffering an acute asthma attack. Longer acting bronchodilators are used to prevent or inhibit the reversible constriction of airway muscle cells. Long-term anti-inflammatories are given to prevent or inhibit the onset of acute asthma attacks. Anti-inflammatory medications include corticosteroids and non-steroidal anti-inflammatory medications intended to relieve the inflammation that can provoke bronchospasm and cause increased mucus. Non-steroidal anti-inflammatory asthma medications are not related to non-steroidal anti-inflammatory drugs (NSAIDs) used for inflammatory conditions such as arthritis.

### SUMMARY

The present invention is based, at least in part, on the discovery that ajulemic acid antagonizes a number of biological activities associated with long-term remodeling of the airway. As described in the Examples below, ajulemic acid (i) decreased the expression of collagenase in fibroblast cells, (ii) inhibited proliferation of airway smooth muscle cells (ASMC), (iii) antagonized the activation of ASMC, and (iv) suppressed calcium signaling in ASMC. Each of these biological activities is associated directly or indirectly with the long term structural changes of airway remodeling. Thus, ajulemic acid can be used in pharmaceutical compositions to prevent or treat one or more features associated with airway remodeling.

In one aspect, the invention features methods of treating airway remodeling in a subject by identifying a subject in need of treatment for airway remodeling; and administering to the subject a pharmaceutical composition including an effective dose of ajulemic acid or a derivative thereof. For example, the subject can have symptoms of asthma; the subject can display symptoms of airway obstruction when the subject is not suffering an asthma attack, or the subject can have a history of chronic asthma; and the subject can display symptoms of airway obstruction when the subject is not suffering an asthma attack. In certain embodiments, the subject (i) has been diagnosed with asthma, (ii) displays symptoms of airway obstruction when the subject is not suffering an asthma attack, or (iii) is being treated with one or more treatments such as an anti-inflammatory, a steroidal anti-inflammatory, or a bronchodilator. In another embodiment, the one or more treatments are administered at their maximal dose. In other embodiments, the subject can have emphysema or can be suffering from chronic obstructive pulmonary disease. In certain embodiments, the pharmaceutical composition includes ajulemic acid.

In another aspect, the invention features methods of treating airway remodeling in a subject by administering a pharmaceutical composition that includes an effective dose of ajulemic acid or a derivative thereof to a subject, wherein the subject is suffering from asthma and is being treated with one or more treatments including an anti-inflammatory, a steroidal anti-inflammatory, or a bronchodilator. In some embodiments, the one or more treatments are administered at their maximal dose. In other embodiments, the subject can be suffering from chronic asthma or severe chronic asthma. In certain embodiments, the pharmaceutical composition includes ajulemic acid.

In other embodiments of the invention, the pharmaceutical composition includes a dose of ajulemic acid of about 1 microgram to about 500 milligrams per kilogram of the subject, about 100 micrograms to about 200 milligrams per kilogram of the subject, about 100 micrograms to about 5 milligrams per kilogram of the subject, or about 200 micrograms to about 2 milligrams per kilogram of the subject.

In various embodiments, the pharmaceutical composition is delivered orally or by inhalation. The pharmaceutical composition can be administered one or more times a day, one or more times a week, one or more times a month, or one or more times a year. The pharmaceutical composition can include a carrier, binder, and/or excipient suitable for oral administration, and a solvent, dispersion medium, and/or propellant suitable for administration by inhalation. The pharmaceutical composition can also be administered with another treatment for asthma.

In another aspect, the invention features pharmaceutical compositions that include ajulemic acid, or a derivative thereof, and a solvent, dispersion medium, and/or propellant suitable for administration by inhalation. In certain embodiments, the invention includes a dispenser containing this pharmaceutical composition, wherein the dispenser delivers a dose for inhalation of ajulemic acid of 1 microgram to about 500 milligrams per kilogram of a subject in need of asthma treatment.

In another aspect, the invention features pharmaceutical compositions that include ajulemic acid, or a derivative thereof, a second therapeutic agent used to treat asthma (e.g., an anti-inflammatory, a steroidal anti-inflammatory, or a bronchodilator), and a pharmaceutically acceptable carrier.

In another aspect, the invention features methods of decreasing the expression of collagenase in fibroblast cells in a subject by administering to the subject a pharmaceutical composition that includes an effective dose of ajulemic acid or a derivative thereof.

In another aspect, the invention features methods of decreasing proliferation of airway smooth muscle cells (ASMC), decreasing ASMC activation, suppressing calcium signaling in ASMC, decreasing NF-KB-mediated expression of target genes, decreasing IK-B phosphorylation, and decreasing matrix metalloproteinase (MMP-1) expression in a subject by administering to the subject a pharmaceutical composition that includes an effective dose of ajulemic acid or a derivative thereof.

Embodiments of the invention can include any combination of features described herein. In no case does the term “embodiment” exclude one or more other features disclosed herein, e.g., in another embodiment.

Ajulemic acid (also referred to as AJA or CT3) is 1′,1′dimethylheptyl-Δ8-THC-11-oic acid.

As used herein, a “subject” refers to human or non-human animal, such as a dog, cat, rodent, bird, horse, cow, pig, sheep, goat, or monkey.

As used herein, “asthmatic” refers to a subject suffering from asthma, or an animal model of asthma.

As used herein, a “maximal dose” of a compound is the largest beneficial dose of the compound. Larger than maximal doses (i) exert no additional therapeutic benefit or (ii) pose an unacceptable risk of side effects.

As used herein, an “effective dose” of a compound is a dose determined to be useful in the treatment of one or more features associated with airway remodeling.

As used herein, “treating” includes affecting the progression or occurrence of an event, e.g., airway remodeling. For example, treating can decrease the progression or occurrence of an event, e.g., airway remodeling.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF DRAWINGS

FIG. 1 is a histogram that quantifies ajulemic acid inhibition of human airway smooth muscle (ASM) cell proliferation.

FIG. 2 is a reproduction of an electrophoretic mobility shift assay (EMSA) showing that ajulemic acid reduces TNF-α induced NF-KB binding to an oligonucleotide corresponding to an NF-KB binding site.

FIGS. 3A and 3B are Western blot images showing that ajulemic acid increases the ratio of unphosphorylated inhibitor protein IK-Bα to phosphorylated IK-Bα in TNF-α stimulated cells.

FIG. 4 is a histogram that quantifies the ability of ajulemic acid to inhibit matrix metalloproteinase (MMP-1) expression.

FIGS. 5A to 5D are a series of graphs and histograms showing that ajulemic acid antagonizes the alteration in calcium homeostasis caused by histamine (FIGS. 5A and 5B) and thrombin (FIGS. 5C and 5D) stimulation of human ASM cells.

### DETAILED DESCRIPTION

Ajulemic acid inhibits a number of biological activities associated with long term remodeling of the airway. Airway remodeling describes a collection of structural alterations that result in persistent airway obstruction. Structural alterations include thickening of the airway wall, decreased resistance and elastance of the airway wall, and exaggerated narrowing of the airway lumen when smooth muscle shortens. The resulting long-term airway obstruction is not reversible by administration of a bronchodilator.

Ajulemic acid antagonizes the biological mechanisms that underlie airway remodeling. Therefore, ajulemic acid can be used to treat or prevent airway remodeling in subjects, e.g., in asthmatics and subjects suffering from emphysema or chronic obstructive pulmonary disease (COPD).

Symptoms, Diagnosis, and Classification of Asthma

Asthma symptoms can include one or more of the following: wheezing, coughing, shortness of breath, and tightness in the chest. These symptoms are especially indicative of asthma if they are recurrent. Episodes of these symptoms, especially coughing, that worsen at night are strongly indicative of asthma.

Airway obstruction can be evaluated using spirometry measurements. Spirometry typically measures (i) forced vital capacity (FVC), and (ii) forced expiratory volume in 1 second (FEV1). The maximal volume of air forcibly exhaled from the point of maximal inhalation, the FEV1, is the volume of air exhaled during the first second of the FVC. Airflow obstruction is indicated by reduced FEV1 and FEV1/FVC values relative to reference or predicted values. The severity of obstruction can be assessed by comparing a subject's spirometric measurements with reference values based on age, height, sex, and race (American Thoracic Society, 1991). Obstructed flow of air from the lungs is indicated by a reduced ratio of FEV1/FVC, i.e., less than 65 percent the lower limit of normal reference value.

Significant reversibility of the obstruction is indicated by an increase of greater than about 12 percent and 200 ml in FEV1 after inhaling a short-acting beta2-agonist bronchodilator, e.g., albuterol or terbutaline. For older subjects, a 2- to 3-week trial of oral corticosteroid therapy may be required to demonstrate reversibility. See generally, Guidelines for the Diagnosis and Management of Asthma, NIH Publication No. 97-4051, National Heart, Lung, and Blood Institute (Bethesda, Md., 1997).

An asthma attack or “exacerbation” results from an acute narrowing of the bronchial airway in a subject. Three main factors can contribute to such a narrowing. The first factor is bronchial inflammation. Inflammation can occur in response to an allergen or irritant that causes bronchial tubes to become red, irritated, and swollen. Inflamed tissues produce fluids that can accumulate and clog the smaller airways. Inflammation can cause tissue damage that leads to sloughing of damaged tissue into the airways, thus further narrowing the airway. The second factor is bronchospasm or tightening of muscles around the bronchial tubes that causes constriction of the bronchial airway. The third factor is hyper-reactivity or hypersensitivity to triggers such as allergens, irritants, and infections. Exposure to triggers can cause or aggravate the inflammation and constriction of the airway.

During an asthma attack, a subject displays a reduced capacity to exhale, which can be assessed using, e.g., the peak expiratory flow rate (PEFR). PEFR measures a value called the peak flow number. During non-severe asthma attacks, peak flow numbers may be in the caution or danger range (usually 50% to 80% of personal best).

Asthma can be classified according to the severity of symptoms in a subject. Severe persistent asthma is characterized by continuous symptoms of asthma, frequent exacerbations, FEV1 or PEFR less than ˜60% of predicted value, and PEFR variability of greater than 30%. Moderate persistent asthma is characterized by daily symptoms, exacerbations more than twice weekly, FEV1 or PEFR from 60% to 80% of predicted value, and PEFR variability greater than 30%. Mild persistent asthma is characterized by the occurrence of symptoms more than twice weekly (yet less frequently than daily), FEV1 or PEFR greater than 80% of predicted values, and a PEFR variability from about 20 to 30%. Mild intermittent asthma is characterized by symptoms that occur, at most, twice weekly, FEV1 or PEFR greater than 80% of predicted values, and a PEFR variability less than 20%.

Typical Treatments for Asthma

Patients with every level of asthma severity are treated with a short-acting bronchodilator (e.g., via an inhaler) to alleviate symptoms of asthma attacks or exacerbations. Short-acting bronchodilators include beta-2 agonists and anticholinergics. Mild intermittent asthma is not treated on a daily basis. Subjects with mild persistent asthma are typically treated daily with an inhaled corticosteroid. Moderate persistent asthma is typically treated with an inhaled steroid anti-inflammatory or a combination of inhaled steroid and a long acting inhaled beta2-agonist bronchodilator. Subjects with severe asthma are prescribed daily corticosteroid pills, long-acting bronchodilators, and/or inhaled steroids. See generally, NIH Practical Guide to the Diagnosis and Management of Asthma, NIH Publication No. 97-4053, National Heart, Lung, and Blood Institute (Bethesda, Md., 1997).

Cromolyn sodium and nedocromil are mild to moderate anti-inflammatory drugs, and may be the initial choice for long-term therapy in children. They are also used preventively, before exercise or before exposure to a trigger. Long-acting beta-2 agonists can also be used just prior to exercise to prevent exercise-induced bronchospasm. Leukotriene modifiers may be considered an alternative to low doses of inhaled corticosteroids or to cromolyn sodium and nedocromil for patients aged 12 years or older with mild persistent asthma. Omalizumab (XOLAIR™) is an injectable immunotherapy for moderate to severe allergic asthmatics for whom corticosteroids fail to control symptoms.

Thus, generally, asthma treatments are directed to (i) preventing or alleviating the inflammatory response that is the major cause of an asthma attack (e.g., with corticosteroids) and/or (ii) relaxing the muscles that surround the bronchial tubes to thereby open the airway.

Ajulemic Acid as Treatment for Remodeling

The methods disclosed herein relate to the use of ajulemic acid to reduce or prevent the persistent, long-term structural changes that occur in the airways of subjects suffering from asthma. Thus, ajulemic acid can be administered (i) on a regular basis as a prophylactic to reduce the likelihood of airway remodeling, (ii) on a regular basis to prevent further airway remodeling, or (iii) to reverse one or more physiological symptoms of airway remodeling. In the methods described herein, ajulemic acid is not administered as a short-acting bronchodilator to treat the symptoms of exacerbation.

Ajulemic acid is 1′,1′dimethylheptyl-Δ8-THC-11-oic acid. This compound is a synthetic analog of the tetrahydrocannabinol (THC) metabolite: 11-carboxy-THC. See Burstein et al., J. Med. Chem., 35:3135-3141, 1992 and Burstein et al., Life Sciences, 63:161-168, 1998.

Generally, the treatment methods described herein include administering a pharmaceutical composition including ajulemic acid to subjects in need of treatment for airway remodeling. Subjects in need of treatment for airway remodeling include those diagnosed with asthma, e.g., mild intermittent, mild persistent, moderate persistent, or severe asthma. Subjects in need of treatment for airway remodeling include subjects who are already being treated with corticosteroids and/or bronchodilator, e.g., with a high dose, moderate dose, or low dose of corticosteroids and/or bronchodilator; including subjects being treated with a maximal dose of corticosteroids and/or bronchodilator.

In some methods, a subject in need of treatment is administered a pharmaceutical composition comprising ajulemic acid on a daily basis, e.g., once, twice, three, or more times a day. In some methods, ajulemic acid is administered on a weekly basis, e.g., once, twice, three, or four times a week, to a subject suffering from asthma. In still other methods, ajulemic acid is administered on a monthly basis, e.g., once, twice, or three times a month, to a subject suffering from asthma.

Pharmaceutical compositions containing ajulemic acid can include dose ranges of from about 1 microgram to about 500 milligrams per kilogram of a subject in need of treatment. Other exemplary dose ranges include from about 100 micrograms to about 200 milligrams per kilogram of a subject, or about 100 micrograms to about 5 milligrams per kilogram of a subject, or about 200 micrograms to about 2 milligrams per kilogram of a subject, or about 100 micrograms to about 1 milligram per kilogram of a subject. For example, a 70 kg subject can be administered a dose of about 140 to 150 micrograms, e.g., 143 micrograms per kilogram. These doses can be administered in the regimes described herein, e.g., once or more per day, once or more per week, or once or more per month. Furthermore, administration of ajulemic acid can continue for months or years, e.g., to prevent the development or worsening of airway remodeling,.

In the treatments described herein, subjects are not administered ajulemic acid in response to symptoms of an asthma attack. Thus, ajulemic acid can be administered to asthmatic subjects who have not recently suffered an asthma attack and/or who are not likely to suffer an asthma attack.

Mechanism of Action

Ajulemic Acid Inhibits Airway Smooth Muscle (ASM) Cell Growth

One mechanism by which ajulemic acid antagonizes airway remodeling is by inhibiting airway smooth muscle (ASM) mass. Hypertrophy and hyperplasia of ASM cells were two of the first recognized features of airway remodeling in asthmatic patients. Postmortem biopsies of patients who died from severe asthma attacks have shown increased size and number of ASM cells throughout the bronchial airway. See, e.g., Carroll et al., Am. Rev. Respir. Dis., 147:405-410, 1993. Prolonged allergen challenges to animal models of asthma have also been reported to cause an increase in ASM mass of the airway. Slamon et al., Eur. Respir. J., 14:633-41, 1999.

Hypertrophy and hyperplasia of ASM cells can also contribute to airway remodeling indirectly, e.g., by amplifying cell signaling events that lead to changes in airway structure. As explained in more detail below, activated ASM cells generate intercellular signals that recruit or activate other cells that contribute to airway remodeling, e.g., by infiltrating the airway and/or changing the extracellular matrix. Thus, by inhibiting ASM cell growth, ajulemic acid reduces or prevents the activated ASM signaling that results in both added cell mass and increased deposition of extracellular matrix in the asthmatic airway.

Example 1, below, describes data showing that ajulemic acid antagonizes proliferation of airway smooth muscle cells.

Ajulemic Acid Antagonizes the Persistent Activation of Airway Cells

When activated, ASM and other airway tissues produce a variety of signaling molecules that contribute to airway remodeling. For example, the transcription factor NF-KB is activated in the sputum and bronchial biopsies of subjects with asthma. See, e.g., Hart et al., Am. J Respir. Crit. Care Med., 158:1585-1592, 1998. Inactive NF-KB is a cytosolic molecule tightly bound to the inhibitory protein IK-B. Signals to activate NF-KB result in the phosphorylation and degradation of IK-B, which exposes a nuclear localization sequence on NF-KB. Once translocated to the nucleus, NF-KB is a potent transcription factor that regulates the release of a variety of potent signaling molecules including tumor necrosis factor-α (TNF-α), interleukins, and chemokines.

These signaling molecules can induce a self-sustaining signaling cascade of growth factors and signaling molecules. In response to signaling molecules (which include, but are not limited to, interleukin-1β (IL-1β), TNF-α, and transforming growth factor-β (TGF-β) airway tissues such as epithelial cells, smooth muscle cells, fibroblasts, and myofibrolasts have been reported to secrete growth factors and other signaling molecules, such as interleukin-1 (IL-1), interleukin-6 (IL-6), epidermal growth factor (EGF), endothelin, and TGF-β. The complicated signaling network is reviewed in Vignola et al., Chest, 123(Supp):417S-422S, 2003. TNF-α has been shown to activate NF-KB and induce IL-1 and IL-6 expression in ASM cells. See McKay et al., Am. J Respir. Cell Mol. Biol., 23:103-111, 2000. Thus, activation of NF-KB in airway tissues initiates a self-sustaining signaling response that maintains airway cells in their activated state and contributes to airway remodeling.

These airway signaling molecules (many of which are NF-KB regulated) are involved in the (i) recruitment, (ii) proliferation, and/or (iii) activation of cells in the airway of asthmatic patients. First, the recruitment and/or localized proliferation of airway cells such as fibroblasts, myofibroblasts, and ASM cells can contribute to the additional tissue mass and abnormal morphology of the remodeled asthmatic airway. Second, fibroblasts and myofibroblasts can further contribute to airway remodeling by releasing components of the extracellular matrix (ECM) such as elastin, fibronectin, and laminin. The abnormal deposition of ECM matrials also contributes to the altered histology and geometry of the airway.

The methods disclosed herein are based, in part, on the discovery that ajulemic acid inhibits the self-sustaining signaling cascade that contributes to airway remodeling. As described in Example 2, below, the administration of ajulemic acid to activated cells inhibits the binding of NF-KB to a target sequence in the transcriptional regulatory region of IK-B. Furthermore, Example 3, below, shows that the administration of ajulemic acid to TNF-α activated cells increased the ratio of inhibitory non-phosphorylated IK-B to phosphorylated IK-B. Thus, ajulemic acid antagonizes NF-KB activity as evidenced by (i) inhibition of NF-KB binding to a target gene for this transcription factor and (ii) by increasing the amount of non-phosphorylated (i.e., NF-KB inhibitory) IK-B present in activated cells.

Thus, one mechanism by which ajulemic acid antagonizes the remodeling phenotype of asthmatic airways is by interfering with the expression of NF-KB-mediated signaling molecules and disrupting the self-sustaining signaling cascade in activated airway cells.

Ajulemic Acid Prevents Thickening and Decreased Elastance of the Airway

Remodeled airways display a thickening and a decreased resistance/elastance of airway walls that correlate with increased expression of specific extracellular matrix (ECM) degradative enzymes, such as collagenases and elastases.

The remodeled airways of asthmatics are thicker due to (i) thickened ECM in the reticular basement membrane than in healthy individuals and (ii) an increased number of cells (e.g., ASM cells and myofibroblasts). See, e.g., Jeffery et al., Am. Rev. Respir. Dis., 145:890-9, 1992. Some studies report that this ECM layer is almost twice as thick in asthmatic subjects (10-15 μm) compared to normal subjects (5-8 μm). For review, see McParland et al., J. Appl. Physiol., 95:426-34, 2003. The increased thickness of this layer appears to correlate directly with an increase in the number of myofibroblasts in the reticular basement membrane, suggesting that myofibrolasts are the source of the additional ECM proteins that contribute to the thickened reticular basement membrane of the asthmatic bronchus. Brester et al., Am. J Respir. Cell Mol. Biol., 3:507-11, 1990. Thus, preventing the infiltration of smooth muscle cells and myofibroblasts (or precursor fibroblasts) into the airway provides a mechanism of reducing the source of both the extra cells or extra ECM proteins that contribute to airway remodeling in asthmatics.

As shown in Example 4 below, ajulemic acid inhibits the production of matrix metalloproteinase 1 (MMP-1), a collagenase, in human ASM cells. While not intending to be bound by theory, one way in which ajulemic acid may antagonize airway remodeling is by reducing expression of ECM degrading enzymes, such as MMP-1, that facilitate migration of ASM cells or myofibroblasts into the airway. This mechanism is consistent with the reported role of MMP-1 in arterial smooth muscle cell systems, where it has been found that MMP-1 inhibitors inhibit SMC migration in vitro and neointima formation in vivo. See e.g., Forough et al., Circulation Res., 79:812-820, 1996. Relatedly, D'Armiento et al., in Cell, 71, 955-961, 1992, demonstrated that transgenic mice over-expressing human MMP-1 in their lungs developed airway morphology changes strikingly similar to the remodeling observed in human pulmonary emphysema. Thus, this mouse model is an accepted animal model of airway remodeling.

Furthermore, overexpression of collagenases and elastases in the airway of asthmatics can reduce the resistance and/or elastance of airway tissues. In normal subjects, the submucosa features a network of long bundles of collagenous elastic ECM fibers composed of collagen and other myofibroblast-derived matrix proteins. Asthmatics, on the other hand, tend to have abnormal, i.e., broken or absent, bronchial elastic and collagen fibers that cause (a) decreased resistance during airway smooth muscle contraction (which facilitates airway narrowing), and (b) decreased elastance which prevents recovery, i.e., widening of the airway after contraction. Thus, diminished resistance and elastance can contribute to the persistent, sustained narrowing of the remodeled airway of asthmatics.

Thus, by inhibiting elastases and collagenases, such as MMP-1, ajulemic acid antagonizes or inhibits the following features of airway remodeling: (i) thickening of the airway due to increased cell mass and/or additional extracellular deposits, and (ii) the loss of airway resistance/elastance due to degradation of collagenous elastic ECM bundles.

Ajulemic Acid Prevents Calcium Transients in ASM cells

TNF-α and other cytokines have been associated with elevated (agonist-induced) cytosolic calcium levels in ASM cells. Altered calcium homeostasis in these cells mediates, or contributes to, intracellular signaling events that regulate one or more of the following functions of ASM cells: (i) increased contractility, (ii) proliferation and/or apoptosis, (iii) migratory function, and (iv) secretion of inflammatory mediators (which include, but are not limited to, interleukin-8 (IL-8) and eotaxin).

As described in Example 5 below, ajulemic acid inhibits an increase in the agonist-induced calcium transients in ASM cells. Thus, ajulemic acid can be used to inhibit features of airway remodeling associated with abnormal calcium homeostasis. For example, ajulemic acid can be used to inhibit or reverse the long-term changes in contractility of ASM cells in asthma patients. Ajulemic acid can also be used to modulate the calcium dependent molecular mechanisms of (i) proliferation and/or apoptosis that results in increased numbers of ASM cells in remodeled airway, (ii) migration that results in the infiltration of additional ASM cells to the airway, and (iii) synthesis and release of pro-inflammatory mediators that can cause lasting injuries to the airways.

Ajulemic Acid Derivatives

Medicinal chemistry can be used to produce derivatives of ajulemic acid. Derivatives can be screened for improved pharmacological properties, for example, efficacy, pharmaco-kinetics, stability, solubility, and clearance. The moieties responsible for ajulemic acid's ability to antagonize the features of airway remodeling can be delineated by examination of structure-activity relationships (SAR) as is commonly practiced in the art. A person of skill in pharmaceutical chemistry could modify moieties on ajulemic acid and measure the effects of the modification on the efficacy of the modified compound to identify derivatives with increased potency. For an example, see Nagarajan et al., J. Antibiot., 41:1430-8, 1998.

Derivatives of ajulemic acid that antagonize one or more symptom of airway remodeling can be used in the methods of treatment described herein. Derivatives of ajulemic acid that can be used include other cannabinoids (see e.g., U.S. Pat. Nos. 5,338,753 and 6,448,288), such as other synthetic analogs of the THC metabolite 11-carboxy-THC, Δ9-THC, and THC-11-oic acid.

Pharmaceutical Compositions

Ajulemic acid and derivatives thereof are referred to herein as active compounds. Active compounds can be incorporated into pharmaceutical compositions. Such compositions typically include the active compound and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. One exemplary route of administration is oral. Oral compositions generally include an inert diluent or an edible carrier. For administration by inhalation, the active compound is delivered in the form of an aerosol spray from a pressurized container or dispenser that contains a suitable propellant, e.g., a gas such as carbon dioxide, or from a nebulizer. For other oral administration, the active compound can be incorporated with excipients and used in the form of tablets, troches, or capsules, e.g., gelatin capsules. Oral compositions can also be prepared using a fluid carrier. Pharmaceutically-compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Other routes of administration include parenteral, e.g., intravenous, intradermal, subcutaneous, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates, and agents for the adjustment of tonicity such as sodium chloride or dextrose. pH can be adjusted with acids, such as hydrochloric acid, or bases, such assodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR ELTM (BASF, Parsippany, N.J.), or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be achieved by including an agent which delays absorption, e.g., aluminum monostearate and gelatin in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient can include a series of administrations of one or more active compound.

Effective dose ranges include milligram or microgram amounts of the small molecule per kilogram of subject or sample weight (e.g., from about 1 microgram to about 500 milligrams per kilogram of a subject in need of treatment. Other exemplary dose ranges include from about 100 micrograms to about 200 milligrams per kilogram of a subject, or about 100 micrograms to about 5 milligrams per kilogram of a subject, or about 200 micrograms to about 2 milligrams per kilogram of a subject, or about 100 micrograms to about 1 milligram per kilogram of a subject). It is furthermore understood that appropriate doses of an active compound depend upon the potency of the compound with respect to the airway remodeling feature to be antagonized. When one or more active compound is to be administered to an animal (e.g., a human) to modulate airway remodeling, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of airway remodeling to be modulated.

The pharmaceutical compositions can be included in a container, pack, or dispenser together with instructions for administration.

Animal Models

The effectiveness of active compounds (i.e., ajulemic acid derivatives) and specific doses of one or more active compounds (e.g., ajulemic acid, a combination of ajulemic acid and a derivative, or a combination of ajulemic acid and another asthma treatment) can be tested in animal models. In one exemplary model, guinea pigs are sensitized to ovalbumin and subsequently exposed to aerosolized ovalbumin, as described in Mirza et al., Clin. Exp. Allergy, 28:644-652, 1998. After repeated exposure to an allergen or vehicle challenge, the airway tissues can be removed and studied for evidence of remodeling. Ohki et al., Pediatric Research, 52:525-532, 2002.

Several rat models for asthma also exist. In virus-induced asthma models, some susceptible (test) and resistant (control) Norway brown rats are administered parainfluenza type-1 virus. See, e.g., Uhl et al., 1996, Am. J. Respir. Crit. Care Med., 154:1834-1842. Allergen-induced models in rats have also been described that are similar to the one described above for guinea pigs. See, e.g., Blyth et al., 1996, Am. J. Respir. Cell Mol. Biol., 14:425-438. Transgenic mouse models of asthma include mice that overexpress IL-6 or IL-11 in murine airway. These animals have been reported to exhibit pronounced airway remodeling phenotypes. DiCosmo et al., J. Clin. Invest., 94:2028-2035, 1996 and Tang, W. et al., J. Clin. Invest. 98:2845-2853, 1996. A dog model of asthma are described in Solway and Fredberg, Am. J Respir. Cell Mol. Biol., 17: 144-146, 1997. Any of these models can be used to test the methods and compositions herein, and are reasonably predictive of human efficiency of the new methods and compositions.

### EXAMPLES

The invention is further described in the following examples, which do not limit the scope of the invention described in the claims.

### Example 1

### Ajulemic Acid Suppression of Human ASM Proliferation

Human airway smooth muscle (ASM) (also known as human bronchial smooth muscle (HBSM)) cells were obtained cryopreserved from Cambrex Bio Science (Walkersville, Md.) at passage 3. See, Ethier and Madison, Cell Calcium, 32:31-38 (2002) and Zhong et al., Am. J Respir Cell Mol. Biol., 30:118-125 (2004). Cultures were characterized as smooth muscle cells by positive immunostaining for smooth muscle actin and negative for Factor VIII. Cells were seeded and grown in smooth muscle basal medium (SMBM, Cambrex Bio Science) supplemented with 5% fetal bovine serum, 5 μg/ml insulin, 0.5 ng/ml epidermal growth factor, 2 ng/ml fibroblast growth factor, gentamicin, and amphotericin. Cells were maintained at 37° C. in a humidified atmosphere (5% CO₂), fed every 48 hours, and passaged when confluent. Shortly after passaging, ajulemic acid (synthesized by Organix, Inc., Woburn, Mass.) was added to growth media to a concentration of 0-30 μmoles. Cells were incubated for 60 minutes, and then stimulated with 1 ng/ml TNF-α for 48 hours. After 48 hours, cell proliferation was evaluated using the Cell Proliferation Kit I (MTT) from Roche Diagnostics (Indianapolis, Ind.), which provides a measure of cell number as a function of optical density.

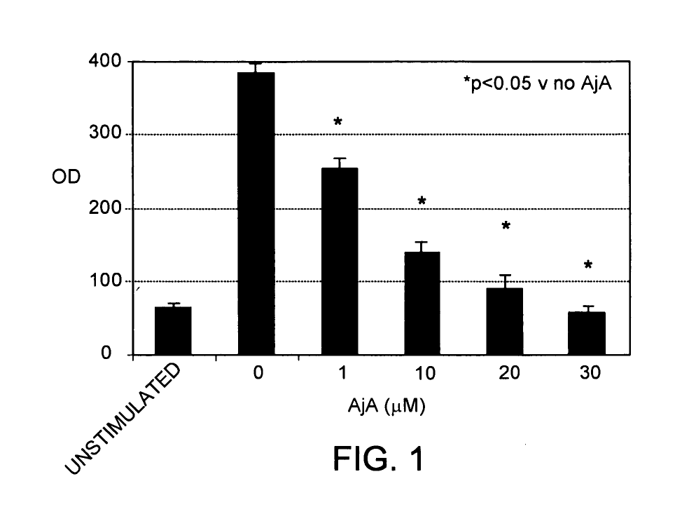

FIG. 1 shows that ajulemic acid suppressed human ASM cell proliferation in a dose-dependent manner. Thus, the data indicate that ajulemic acid can be used in a therapeutic composition to inhibit the proliferation of smooth muscle cells in the airway of asthmatics.

### Example 2

### Ajulemic Acid Reduces TNF-α Induced NF-KB Activation

Human fibroblast-like synovial (FLS) cells were isolated from synovial tissue or synovial fluid of patients with inflammatory arthritis (4 RA, 1 psoriatic arthritis) essentially as described (Stebulis et al., J. Rheumatol. 32:301-06, 2005). Briefly, synovial tissue obtained at the time of joint replacement surgery was minced and plated in tissue culture dishes with growth medium (MEM with 15% FBS, 1% penicillin/streptomycin solution, 1% nonessential amino acids). Synovial fluid was collected in heparinized syringes, then centrifuged at 1200 rpm for 15 min. The cell pellet was resuspended in growth medium, then plated in tissue culture flasks. After 2-4 days, primary cultures were washed with PBS to remove tissue fragments and nonadherent cells. FLS were incubated at 37° C. under 5% CO₂ and passaged to 6-well tissue culture plates (split 1:3) when they reached confluence. Passages 2 through 8 were used for experiments.

Experiments were done on FLS rested overnight in low serum medium (MEM/2% FBS). Medium was replaced before beginning the experiments. Cells were treated with 10 μM ajulemic acid for 1 hour then stimulated with 1 ng/ml TNF-α or left unstimulated. Controls were vehicle (0.3% DMSO) treated cells. Nuclear extracts were prepared 45 minutes after stimulation and assayed for binding activity to NF-KB binding sites by electrophoretic mobility shift assays (EMSA). To perform the EMSA, oligonucleotides (5′-GGGACTTTCC-3′) corresponding to a wild type NF-KB binding site on gene plasmids (Celgene Signal Research Division, San Diego, Calif.) were synthesized using a Beckman 1000 M DNA synthesizer and labeled with ³²P using T4 polynucleotide kinase (New England BioLabs, Beverly, Mass.). Nuclear extracts were prepared and dNA binding reactions were performed using 10 μg of nuclear extract, 40 kcpm of probe, 7.1-10 μl KN-100 (1M HEPES [pH 7.9], 1M KCL, 0.5M EDTA, 100% glycerol), and 1 μg poly(dl-dC)·poly(dl-dC). The reactions were incubated for 30 minutes. Equal aliquots of each sample were loaded onto a 4% polyacrylamide gel and electrophoresed at 200V for 1.5-2 hours at 4° C. Gels were dried and autoradiography wasperformed by exposure to KODAK® Xomat film. Gels were scanned and analyzed using ADOBE® software for PC.

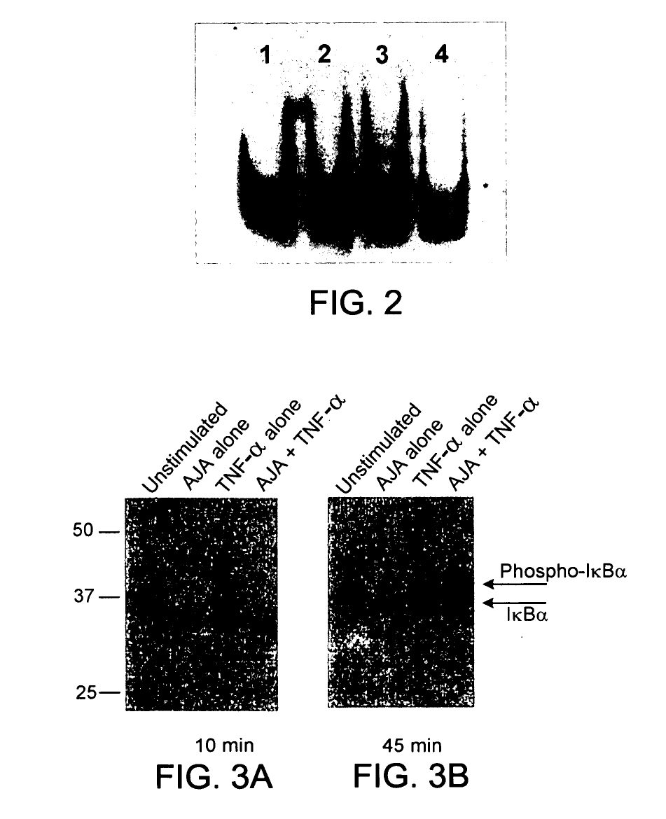

As shown in FIG. 2, nuclear binding of NF-KB is not altered in resting FLS treated with ajulemic acid (lane 2 vs. lane 1). However, ajulemic acid did reduce TNF-α induced NF-KB activation (as measured by binding to the oligonucleotide corresponding to an NF-KB binding site) by approximately 75% (lane 4 vs. lane 3). Competition binding with cold probe eliminated the effect (data not shown).

To identify and confirm expression of the subunits of NF-KB, supershift assays are performed. Antibodies to the subunits of interest (e.g., P65 heterodimer, P50 homodimer subunits) are added to the protein/probe complex before application to the acrylamide gel in the EMSA assay. Samples are incubated for 1 hour or up to overnight at 4° C. Antibodies to NF-KB subunits are readily available (Santa Cruz Biotechnology). The complex of antibody/protein/probe retards the migration on the electrophoresis gel and thus a ‘supershift’ results. Other aspects of the supershift assay, including preparation of nuclear extracts, NF-KB oligonucleotide labeling and binding reaction, polyacrylamide gel electrophoresis and visualization of samples by autoradiography are identical to the EMSA assay.

These results suggest that ajulemic acid can be used to antagonize NF-KB-mediated expression of target genes.

### Example 3

### Ajulemic Acid Inhibits TNF-α Signaling by Preventing Phosphorylation of IK-B

FLS cells were collected from human synovial fluid in heparinized syringes, then centrifuged at 1200 rpm (260 g) for 15 minutes. FLS cells are used herein as models of pulmonary fibroblasts and/or myofibroblasts. The resulting cell pellet was resuspended in 7 ml of growth medium (Dulbecco's Minimal Essential Medium with 15% heat-inactivated fetal bovine serum (FBS), 1% nonessential amino acids, 1% penicillin/streptomycin solution) and plated in 25 ml tissue culture flasks. Cultures were incubated at 37° C., under 5% CO₂ for 24 to 48 hours after which medium was aspirated and cultures were washed with PBS to remove nonadherent cells. Growth medium was replaced every 3 to 4 days. After 10 to 14 days, adherent cells were harvested by trypsinization, washed, and transferred to 6-well tissue culture plates in fresh growth medium. FLS cells were passaged (split 1:3) at confluence, generally every 10 to 14 days.

FLS cells at passage 2 through 6 were (i) untreated, (ii) treated with 1-30 μM of ajulemic acid in growth media, (iii) treated with 1 ng/ml of TNF-α in growth media, or (iv) treated with a combination of 1-30 μM of ajulemic acid and 1 ng/ml TNF-α for 10 minutes or 45 minutes. Cells were harvested and washed several times with PBS. Whole cell lysates were prepared in hypotonic HEPES buffer (7.45 g KCl/100 ml H₂O; 3.80 g EGTA/100 ml H₂O;0.87 g PMSF/500 ml H₂O; 1.54 g DTT/10 ml H₂O). IK-β was immunoprecipated by mixing whole cell lysates with a recombinant antibody to IK-β from Santa Cruz Biotechnology, Inc. (Santa Cruz, Calif.) and incubating for 2 hours at 4° C. Immunocomplexes were collected by adding Protein A agarose for 1 hour. Samples were then centrifuged. Pellets were washed and then boiled for 5 minutes. Cell debris was pelleted by short spin in microcentrifuge. Supernatant sample was collected and prepared by adding an equal amount of 2× Laemmli buffer. Samples were then applied to 10% SDS-PAGE gel. Electrophoresed samples were transferred to nitrocellulose membranes, which were blocked with 2% casein, and probed with primary Ab to IK-β from Santa Cruz Biotechnology, Inc. (Santa Cruz, Calif.) and then secondary antibody conjugated to alkaline phosphatase (Tropix, Bedford, Mass.) added. Secondary antibody was visualized using a chemiluminescent alkaline phosphatase substrate and radiography.

FIG. 3 shows that stimulation with TNF-α (for both 10 and 45 minutes) resulted in an increase in the phosphorylated form of IK-B, which is not inhibitory to NF-KB and should, therefore, upregulate expression of NF-KB-regulated genes. However, treatment with ajulemic acid (i) prevented phosphorylation of IK-B (phosphor-IK-B) 10 minutes after stimulation with TNF-α and (ii) dramatically increased expression of the non-phosphorylated form of IK-B after 45 minutes.

These results are consistent with a mechanism for ajulemic acid anti-remodeling activity by which ajulemic acid inhibits TNF-α activation of NF-KB, which in turn prevents NF-KB mediated expression of signaling molecules. In this manner, ajulemic acid can antagonize the expression of molecules that cause or exacerbate airway remodeling in asthmatics.

### Example 4

### Ajulemic Acid Inhibits Collagenase Expression in Human FLS Cells

Human fibroblast-like synovial cells (FLS) were obtained from synovial tissue or synovial fluid, and grown to confluence in 6 well tissue culture plates. Cells were treated or not for 60 minutes with AjA, then stimulated with 10 ng/mL TNFα+1 ng/mL GMCSE for 4 hours. MMP-1 mRNA content measured by Northern blot analysis.

Cells were harvested and resuspended in TRIZOL® solution (GIBCO-BRL) to extract total cellular RNA. The RNA was resuspended in diethylpyrocarbonate (DEPC)-treated water. Total RNA (15 μg) was separated by electrophoresis in 1% agarose/17.6% formaldehyde gels. The RNA was transferred overnight from the denaturing gel onto Hybond-N⁺ (Amersham Pharmacia Biotech Inc., Piscataway, N.J.) by capillary action using 10× SSC buffer (3M NaCl, 0.3 M sodium citrate). The RNA was cross-linked to the filter by ultraviolet irradiation for 20 seconds using a UV Crosslinker (Spectronics Corporation, Westbury, N.Y.) and the blots were hybridized with a solution of 5×SSC, 5× Denhardt's solution, 0.5% SDS, and 10 mg/ml sonicated salmon sperm DNA for 5 hours at 65° C., and 10⁷ cpm/ml radio-labeled cDNA generated by random priming with ³²P-dCTP (30,000 Ci/mmol, NEN, Boston, Mass.). Probes specific for the MMP-1 gene were heat denatured and added to the prehybridization buffer for hybridization overnight at 65° C. The RNA blot was washed in buffers of progressive stringencies starting at 2×SSC (0.1% SDS), followed by 1×SSC and a final rinse in 0.1×SSC (0.1% SDS) at 65° C. The blot was subjected to autoradiographic exposure overnight at −70° C.

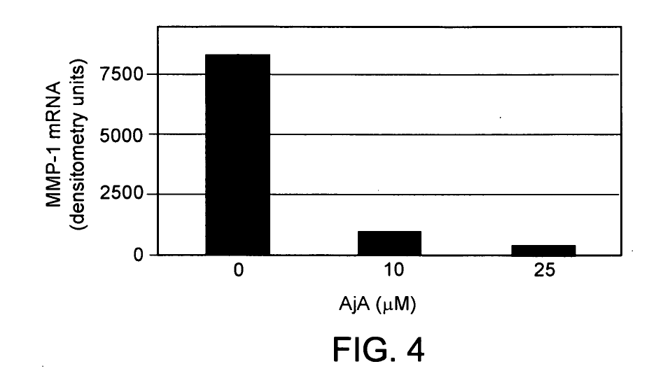

As quantified in the graph of FIG. 4, MMP-1 Northern blot results revealed that ajulemic acid downregulates MMP-1 expression in a dose-dependent manner. These data indicate that ajulemic acid can antagonize changes in ECM that can lead to or exacerbate the following features of airway remodeling that are associated with increased MMP-1 collagenase expression: increased cellular infiltration to the airway and decreased airway resistance/elastance.

### Example 5

### Ajulemic Acid Inhibits Calcium Transients

Human airway smooth muscle (ASM) cells were obtained and passaged as described in Example 1. Subconfluent ASM cells in 96-well plates were washed 3 times in a modified Kreb-Ringer-Henseleit solution (KRH) containing 115 mM NaCl, 5 mM KCl, 1 mM KH2PO₄, 1 mM MgSO₄, 25 mM N-2-hydroxyethylpiperazine-N′-2-ethanesulfonic acid (HEPES), 15 mM glucose and 2 mM CaCl₂. Cells were incubated in 1.0 μM fura 2-AM and pluronic F-127 (0.004%) for 60 minutes at room temperature. See, e.g., Wang et al., Am. J. Phys.; 272:C1151-C1159, 1997. After washing 3 times with KRH, cells were covered with 200 μl KRH and placed on the microscope stage. Reagents were added directly to the wells during calcium recordings. Fura 2-AM and F-127 were from Molecular Probes, Inc. (Eugene, Oreg.). Other chemicals were from Sigma (St. Louis, Mo.).

Fluorescence was induced using computer-controlled 337 and 380 nm UV light generated by a nitrogen laser and a nitrogen laser-pumped dye laser, respectively (Laser Science, Franklin, Mass.). See, e.g., Ethier et al., Cell Calcium, 32:31-38 2002. Each laser alternately fired pulses at 30 Hz and these alternating pulses of light were guided by a bifurcated quartz fiber to a neutral density filter at the epiport of the microscope and then focused on cells through a 40× objective lens. Fluorescent signals emitted by fura-2 were passed back through the objective to a 455 nm dichroic mirror, a 475 nm barrier filter (Omega Optics, Brattleboro, Vt.), an image intensifier (Xybion Electronic Systems, San Diego, Calif.) and captured by a Philips-based frame transfer charge coupled device (CCD) camera (CCTV, New York, N.Y.). The analog signals from the camera were digitized and stored in an imaging board. Digital outputs were transferred to a personal computer with software by Recognition Technology, Inc. (Framingham, Mass.).

Background from a cell free region of the cover glass was subtracted prior to data acquisition and then an 11×11 pixels area was selected over each cell. Areas of the cell containing the nucleus were avoided. Fluorescence emissions stimulated by alternating pulses of ˜340 and 380 nm light were recorded and their ratios plotted. Ratios were converted to calcium concentrations using [Ca²⁺]i=KD·β·(R-Rmin)/(Rmax-R), as previously reported in Grynkiewicz et al., J. Biol. Chem., 260:3440-3450, 1985.

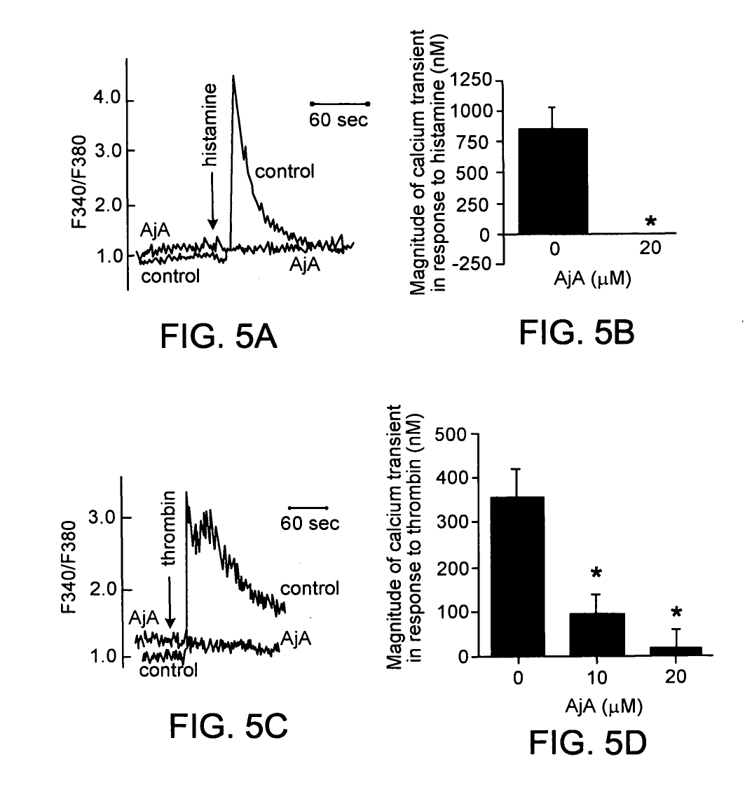

FIGS. 5A to 5D show that ajulemic acid suppressed calcium transients in human ASM cells stimulated with either histamine or thrombin. To generate the results shown in FIGS. 5A to 5D, ASM cells were treated with ajulemic acid (10 μM or 20 μM) for 10 minutes (or were left untreated to serve as controls) and exposed to histamine (FIGS. 5A and 5B) or thrombin (FIGS. 5C and 5D). FIGS. 5A and 5C show the F340/F380 ratio (a measure of cytosolic calcium concentrations) over a time course of histamine or thrombin treatment, respectively. These results are presented in graphic form in FIGS. 5B and 5D, respectively. The effects of ajulemic acid on calcium transients are statistically significant, as indicated by the * in FIGS. 5B and 5D (*p<0.01 for ajulemic acid-treated cells versus control cells, as determined by ANOVA (n=4 to 9)).

These results indicate that ajulemic acid can be used to antagonize calcium-mediated events in ASM cells that lead to or exacerbate airway remodeling. For example, ajulemic acid can be used to inhibit one or more of the following calcium-mediated events in the airway (i) increased ASM contractility, (ii) ASM proliferation and/or apoptosis, (iii) ASM migration and infiltration of the airway, and (iv) secretion of inflammatory mediators (which include, but are not limited to, IL-8 and eotaxin).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating airway remodeling in a subject, the method comprising
identifying a subject in need of treatment for airway remodeling; and
administering to the subject a pharmaceutical composition comprising an effective dose of ajulemic acid or a derivative thereof.

2. The method of claim 1, wherein the subject has symptoms of asthma; and the subject displays symptoms of airway obstruction when the subject is not suffering an asthma attack.

3. The method of claim 1, wherein the subject has a history of chronic asthma; and the subject displays symptoms of airway obstruction when the subject is not suffering an asthma attack.

4. The method of claim 1, wherein the subject (i) has been diagnosed with asthma, (ii) displays symptoms of airway obstruction when the subject is not suffering an asthma attack, and (iii) is being treated with one or more treatments selected from the group consisting of an anti-inflammatory, a steroidal anti-inflammatory, and a bronchodilator.

5. (canceled)

6. The method of claim 1, wherein the subject has emphysema.

7. The method of claim 1, wherein the subject is suffering from chronic obstructive pulmonary disease.

8. The method of claim 1, wherein the pharmaceutical composition comprises ajulemic acid.

9. The method of claim 1, wherein the subject is suffering from asthma and is being treated with one or more treatments selected from the group consisting of an anti-inflammatory, a steroidal anti-inflammatory, and a bronchodilator.

10. (canceled)

11. The method of claim 9, wherein the subject is suffering from chronic asthma.

12. (canceled)

13. The method of claim 9, wherein the pharmaceutical composition comprises ajulemic acid.

14. The method of claim 1, wherein the pharmaceutical composition comprises a dose of ajulemic acid of 1 microgram to about 500 milligrams per kilogram of the subject.

15. The method of claim 1, wherein the pharmaceutical composition is delivered orally.

16. The method of claim 1, wherein the pharmaceutical composition is delivered by inhalation.

17. The method of claim 1, wherein the pharmaceutical composition is administered one or more times a day.

18-19. (canceled)

20. The method of claim 1, wherein the pharmaceutical composition is administered with another treatment for asthma.

21. A pharmaceutical composition comprising ajulemic acid, or a derivative thereof, and one or more of a solvent, dispersion medium, and propellant suitable for administration by inhalation.

22. A dispenser comprising the pharmaceutical composition of claim 21, wherein the dispenser delivers a dose for inhalation of ajulemic acid of 1 microgram to about 500 milligrams per kilogram of a subject in need of asthma treatment.

23. A pharmaceutical composition comprising ajulemic acid, or a derivative thereof, a second therapeutic agent used to treat asthma, and a pharmaceutically acceptable carrier.

24-30. (canceled)

31. The method of claim 14, wherein the dose is about 100 micrograms to about 200 milligrams per kilogram of the subject.

32. The method of claim 14, wherein the dose is about 100 micrograms to about 5 milligrams per kilogram of the subject.

33. The method of claim 14, wherein the dose is about 200 micrograms to about 2 milligrams per kilogram of the subject.

34. The method of claim 1, wherein the pharmaceutical composition is administered one or more times a week.
